(19) United States
(12) Patent Application Publication
(10) Pub. No.: US 2017/0028164 A1
(43) Pub. Date: Feb. 2, 2017

(54) Mobile Work Stress Relief

(71) Applicant: Meditation Works, LLC, Huntington Woods, MI (US)

(72) Inventor: Jacob Leider, Huntington Woods, MI (US)

(21) Appl. No.: 15/186,659

(22) Filed: Jun. 20, 2016

Related U.S. Application Data
(60) Provisional application No. 62/198,808, filed on Jul. 30, 2015.

(51) Int. Cl. A61M 21/00; G06Q 10/06; A61M 21/02; B62D 63/06

(52) U.S. Cl. CPC A61M 21/0094; B62D 63/06; G06Q 10/06314; A61M 21/02; H04W 88/02

(57) ABSTRACT

A mobile meditation unit has a scheduling control for receiving a request for a mobile meditation session. The control is programmed to receive a request, and schedule a mobile meditation unit to be dispatched to a particular location. A mobile meditation unit includes an enclosed space having climate control and seating locations. A method of providing a mobile meditation system is also disclosed.

### RELATED APPLICATION

This application claims priority to U.S. Provisional Application No. 62/198,808, filed Jul. 30, 2015.

### BACKGROUND

This application relates to a method and system for providing mobile stress relief techniques through meditation.

Work places are becoming increasingly stressful for modern workers. Text messages, e-mail notifications, and telephone calls disrupt concentration and add to the stress levels.

Progressive companies often provide traditional wellness programs for their employees but gym memberships, etc. demand a good deal of time to be useful, and actually sometimes increase stress levels even more.

Meditation is a known way of addressing stress, however, the demands of modern life make it difficult to successfully meditate, especially in a work environment.

### SUMMARY

A mobile meditation unit has a scheduling control for receiving a request for a mobile meditation session. The control is programmed to receive a request, and schedule a mobile meditation unit to be dispatched to a particular location. A mobile meditation unit includes an enclosed space having climate control and seating locations.

A method of providing a mobile meditation system is also disclosed.

These and other features may be best understood from the following drawings and specification.

### BRIEF DESCRIPTION OF THE DRAWINGS

FIG. 1 schematically shows a system according to this disclosure.

### DETAILED DESCRIPTION

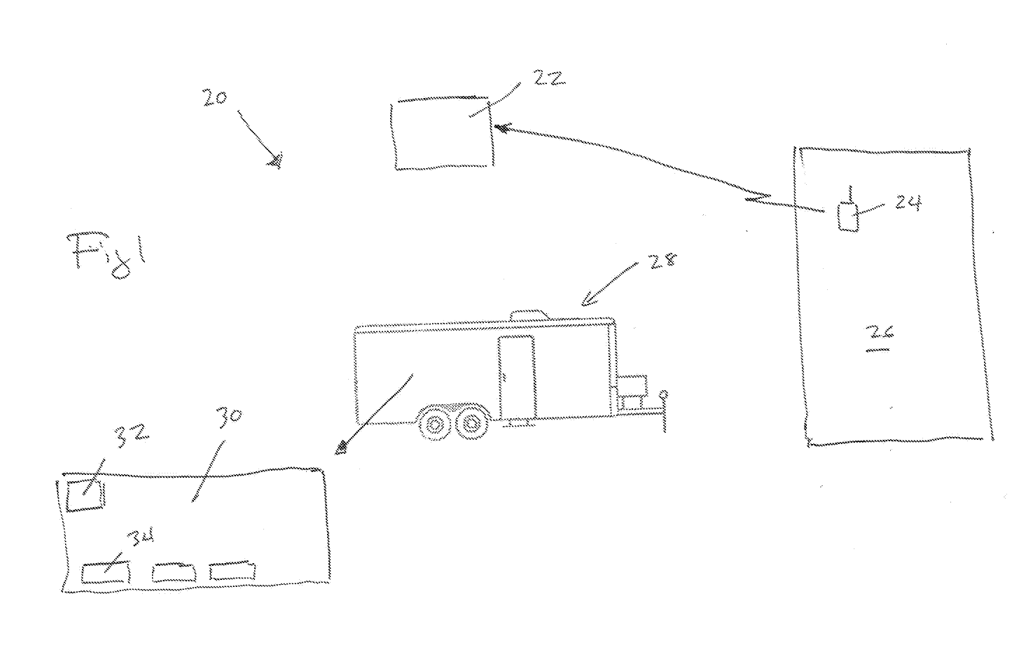

FIG. 1 shows a system 20 for providing a mobile meditation unit. A central control system 22 may communicate with a device such as a cell phone 24 to allow a company to order a visit from a mobile meditation unit to a work space such as building 26. Control unit 22 will then dispatch the mobile unit 28. The control unit 22 may be any type of computer, or other appropriate system.

Alternatively, a mobile unit 28 could appear upon a pre-arranged schedule.

There may be various options for different meditation styles that could be provided, and a mobile application may be provided to allow the phone 24 to select a particular one of those options.

The mobile unit 28 should provide a stress relieving environment. As such, an interior 30 of the mobile unit is climate controlled at 32. Further, it is provided with sitting locations 34.

A supervisor at the work space 26 will identify a need to provide stress management for the employees at the work space. The mobile meditation unit 28 will then be dispatched to the work space.

Workers can then move into the interior 30, and be provided with a quiet space in which to practice meditation.

A leader will provide a (say 10 to 12 minutes long) guided meditation session. This could be a pre-recorded session. The participants will not be allowed to bring any electronic devices into the mobile meditation unit 28.

The leader will lead participants through focused breathing, relaxation and awareness steps. The details of the mediation may be as generally known.

Employees will be able to return to work (say within 15 minutes), having enjoyed physical and mental health benefits. Those benefits include but are not limited to decreased heart rate, decreased blood pressure, increased focus, increased logic, decreased reactivity response, increase compassion and empathy, lower stress levels and improved circulation.

While an embodiment has been disclosed, a worker of ordinary skill in this art would recognize that modifications would come within the scope of this disclosure. For that reason, the following claims should be studied to determine the true scope and content of this disclosure.

1. A mobile meditation unit comprising:
a scheduling control for receiving a request for a mobile meditation session, said control being programmed to receive a request, and schedule a mobile meditation unit to be dispatched to a particular location; and
a mobile meditation unit including an enclosed space having climate control and seating locations.

2. The unit as set forth in claim 1, wherein a mobile communication device sends the request for a meditation session.

3. The unit as set forth in claim 3, wherein an application is provided on the mobile communication device to send the request.

4. A method of providing a mobile meditation system including the steps of:
receiving a request for a mobile meditation session at a control, said control scheduling and dispatching a mobile meditation unit to a particular location; and
the mobile meditation unit including an enclosed space having climate control and seating location.

5. The method as set forth in claim 4, wherein a mobile communication device sends the request for a meditation session.

6. The method as set forth in claim 5, wherein an application is provided on the mobile communication device to send the request.
